(19) United States
(12) Patent Application Publication
(10) Pub. No.: US 2010/0184659 A1
(43) Pub. Date: Jul. 22, 2010

(54) Sustained-Release Formulations Comprising Crystals, Macromolecular Gels, and Particulate Suspensions of Biologic Agents

(76) Inventor: Warren Jaworowicz, Bolton, MA (US)

Correspondence Address: K&L Gates LLP, STATE STREET FINANCIAL CENTER, One Lincoln Street, BOSTON, MA, 02111-2950, US

(21) Appl. No.: 12/516,922

(22) PCT Filed: Dec. 19, 2007
(86) PCT No.: PCT/US07/25956

§ 371 Date: May 29, 2009

Related U.S. Application Data
(60) Provisional application No. 60/876,292, filed on Dec. 21, 2006.

(51) Int. Cl. A61K 38/16; C07K 14/00; A61P 19/02; A61P 1/02; A61P 3/14; A61P 9/10; A61P 25/00; A61P 25/16; A61P 27/02

(52) U.S. Cl. 514/12; 530/350

(57) ABSTRACT

The present invention is directed to sustained release formulations of biologic agents which permit persistent bioavailability. Preferred biologic agents include bone morphogenetic proteins. Diseases susceptible to amelioration and/or treatment with the formulations of the present invention include skeletal tissue diseases such as, but not limited to, osteoarthritis and other osteochondral diseases. The sustained release formulations of the present invention are especially suitable for treatment of minimally-vascularized or non-vascularized tissue sites such as, but not limited to, intra joint, interarticular, or intraminiscal sites.

### CROSS-REFERENCE TO RELATED APPLICATIONS

This application claims priority to and the benefit of U.S. Provisional Patent Application No. 60/876,292, filed Dec. 21, 2006, the contents of which are incorporated by reference herein.

### TECHNICAL FIELD

The invention generally relates to sustained-release formulations for the delivery of biologic agents (BA), more specifically proteins; even more specifically proteins with low physiological solubility; and especially bone morphogenetic proteins (BMPs). The formulations are compositions comprising solid or liquid BA crystals (both with and without the crystallization solvent), BA macromolecular gels, or BA particulate suspensions. The invention further provides pharmaceutical compositions as well as methods of administering the above-described formulations and pharmaceutical compositions systemically or directly to tissues, particularly joints impacted by disease, especially osteoarthritis and osteochondral disease. Additionally, the invention is directed to kits comprising the aforementioned formulations and compositions for use in the treatment of disease, particularly osteoarthritis and osteochondral disease. The invention also relates to methods for treating injury or disease with solid and liquid BA crystals, BA macromolecular gels, and BA particulate suspensions.

### BACKGROUND

Bone morphogenetic proteins (BMPs) belong to the superfamily of transforming growth factor β (TGF-β), and control a diverse set of cellular and developmental processes, such as pattern formation and tissue specification as well as promoting wound healing and repair processes in adult tissues. BMPs were initially isolated by their ability to induce bone and cartilage formation. BMP signaling is inducible upon bone fracture and related tissue injury, leading to bone regeneration and repair.

To date, a reliable means for delivering a clinically effective dose of a BMP over a prolonged period of time, without repeated administration of the BMP, has heretofore eluded the skilled practitioner. In fact, sustained delivery of proteinaceous BAs generally remains an unanswered challenge. Moreover, despite progress in protein technologies and pharmaceutical chemistries, at least two problems continue to plague clinicians needing to provide sustained levels of key physiological factors to patients.

First, most therapeutic agents are administered orally. However, oral administration and other conventional drug delivery methods often are inappropriate for macromolecular drugs, as many of them are unstable in the blood stream and/or gastrointestinal tract, are toxic at high doses or have a narrow therapeutically effective concentration range (therapeutic window). This is further complicated in the case of chondral or osteochondral diseases and/or diseases or injuries of the joint since such tissues are poorly vascularized and not susceptible to treatment using some routine modes of systemic administration. Additionally, therapeutic proteins, for example, are typically administered by frequent injection because proteins generally have short in vivo half-lives and/or negligible oral bio-availability. This poses a substantial physical burden on the patient and creates significant administrative costs related to patient management. To provide greater efficacy, safety, patient convenience, and patient compliance, much effort has been spent attempting to develop and evaluate improved sustained-release formulations for protein and other macromolecular drugs. At the very least, a sustained release modality which permits sustained local release via a single administration would be desirable.

Second, formulations that obviate the need for the active ingredient to be prepared with a carrier, vehicle, or other inactive agents eliminate a great deal of the complexity inherent in manufacturing a dosage form. Other benefits of such comparatively simple dosage forms include lower manufacturing costs as well as the potential for higher active yields. Thus a modality that does not require carriers, vehicles, or other inactive agents would provide the skilled artisan with a preferable alternative means for administering biologically active agents systemically or locally.

Thus, there is a need for additional sustained delivery formulations suitable for administering biologically active agents, especially macromolecules such as BMPs and other proteinaceous macromolecular biologics or drugs.

### SUMMARY OF THE INVENTION

The present invention is based on the discovery that the higher order three-dimensional architecture or tertiary structure of a BA, especially proteins in general, can be exploited when preparing sustained or timed release formulations. By preserving these higher order structures, a depot of BA can be prepared from which individual protein molecules are released over time and become biologically available and functional. Moreover, a limiting factor to date for optimal use of proteins, particularly in therapeutic regimens, has been the sensitivity of an individual protein's structure to chemical and physical denaturation encountered during medicament manufacture and subsequent delivery. The present invention can obviate such limitations. Another limiting factor relates to bioavailability and its dependence upon the choice of mode of administration, i.e., systemic versus local administration which is particularly so in the case of tissues or tissue sites having a diminished or negligible blood supply, such as for example a non-mineralized skeletal tissue such as cartilage. The present invention allows the skilled artisan to provide a persistently bioavailable dose of a biologic agent either locally, i.e. implantation, or systemically, i.e., subcutaneously or intramuscularly.

The present invention is directed to compositions comprising a solid or liquid BA crystal, BA macromolecular gel, or BA particulate suspension wherein the BA is released in an effective amount. “Macromolecular gel” as used herein does not refer to the use of carrier gels, such as PLG-PEG, or similar polymer compositions. Rather, “macromolecular gel” refers to a state of gelation and/or a gelation phenomenon attributable to the macromolecular ordering of the BA per se. The present invention provides such BA compositions that are released in a sustained release manner. The present invention also provides such BA compositions that are particularly suitable for implantation at a tissue site. In another aspect of the present invention, the tissue site is vascularized. In one embodiment of the present invention, the tissue site is non-vascularized. In a further embodiment, the tissue site is a joint. In a further embodiment, the tissue site is the inter-articular space. In another aspect of the present invention, BA compositions that are suitable for systemic administration are provided. In one embodiment, the systemic administration is either subcutaneous or intramuscular. In another aspect, the present invention features a BA crystal, macromolecular gel, or particulate suspension composition in which the BA is proteinaceous. In one embodiment, the proteinaceous BA is a minimally soluble protein. In one embodiment, the proteinaceous BA is a protein that is substantially insoluble at physiological pH. In one embodiment, the proteinaceous BA is a member of the TGF-β superfamily of proteins. Another embodiment of the present invention provides for a proteinaceous BA that is a member of the BMP subfamily of the TGF-β superfamily of proteins. In one embodiment of the present invention, the proteinaceous BA is BMP-2 (SEQ ID NO:1), BMP-4 (SEQ ID NO:3), BMP-5 (SEQ ID NO:7), BMP-6 (SEQ ID NO:9), BMP-7 (SEQ ID NO:11), GDF-5 (SEQ ID NO:13), GDF-6 (SEQ ID NO:15) and GDF-7 (SEQ ID NO:17). In another aspect of the present invention, the proteinaceous BA is BMP-7. The present invention also provides for a proteinaceous BA that is sequence variant of any one of BMP-2, BMP-4, BMP-5, BMP-6, BMP-7, GDF-5, GDF-6, or GDF-7. In another aspect of the present invention, the proteinaceous BA is a protein having at least about 50% amino acid sequence identity with a member of the BMP subfamily within the conserved C-terminal cysteine-rich domain.

The present invention further provides for compositions wherein the BA crystal, macromolecular gel, or particulate suspension is formed ex vivo. In another aspect of the invention, the BA composition further comprises a release modifying agent. In another embodiment, the BA composition further comprises a bulking agent. The present invention also provides for BA compositions in amount effective to ameliorate tissue injury or disease. In one embodiment, the injury to be ameliorated is a mineralized or non-mineralized skeletal tissue injury. In another embodiment, the injury or disease to be ameliorated is metabolic bone disease, osteoarthritis, osteochondral disease, rheumatoid arthritis, osteoporosis, Paget's disease, periodontitis, dentinogenesis, chondral disease, trauma-induced and inflammation-induced cartilage degeneration, age-related cartilage degeneration, articular cartilage injuries and diseases, full thickness cartilage diseases, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, or injuries and diseases of ligament, tendon, synovial capsule, synovial membrane and meniscal tissues. In another embodiment, the injury or disease to be ameliorated is liver disease, liver resection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, or ulcerative diseases of the gastrointestinal tract.

Methods of the present invention comprise the steps of providing systemically, or locally, a composition comprising a BA crystal, BA gel, or BA particulate suspension in an amount effective to treat injury or disease. In one embodiment of the invention, the BA composition is suitable for implantation. In another embodiment, the BA composition is provided either subcutaneously or intramuscularly. In another aspect of the invention, the method comprises the step of providing the BA composition to a vascularized tissue site. In another embodiment, the method comprises the step of providing the BA composition to a non-vascularized tissue site. In one aspect of the invention, the BA composition is implanted in the inter-articular space. The methods of the present invention also provide for a BA composition whose release is sustained for at least 2-7 days. In another aspect of the methods of the present invention, the BA composition is provided in an effective amount of about 10 to 1000 micrograms for the treatment of osteoarthritis. The present invention also provides pharmaceutical compositions and kits comprising any of the compositions disclosed above.

The foregoing, and other features and advantages of the invention as well as the invention itself, will be more fully understood from the following figures, description, and claims.

### BRIEF DESCRIPTION OF FIGURES

FIG. 1 comprises photographs at 1, 5, 22, and 96 hours (from left to right) of a BMP-7 crystal transferred into 50 mM acetic acid (pH 4) at room temperature.

FIG. 2 comprises photographs at 1, 5, 22, and 96 hours (from left to right) of a BMP-7 crystal transferred into phosphate buffered saline (PBS) at room temperature.

FIG. 3 comprises photographs at 1, 5, 22, and 96 hours (from left to right) of a BMP-7 crystal transferred into bovine synovial fluid at room temperature.

FIG. 4 comprises a photograph of a high concentration protein gel of BMP-7 right after its production by centrifugal concentration in 50 mM acetic acid.

FIG. 5 comprises a photograph of a high concentration protein gel of BMP-7 after 24 hours of rocking in 50 mM acetic acid at 37 degrees Celsius.

### DETAILED DESCRIPTION

The present invention is based on the discovery that BMPs, such as BMP-7 which is an exemplary BMP, can be formulated to provide a sustained release composition having ameliorative and restorative effects on injured, diseased or damaged cartilage without an associated inflammatory or irritative response at the site of intra-joint or intraminiscal administration. According to the present invention, a composition is provided in which a BA, preferably a proteinaceous agent and most particularly a BMP, is in a solid or liquid crystalline form, or as a macromolecule gel or particulate suspension, with or without solvents or release-modifying agents. When administered, the composition of the present invention provides a sustained release depot of the BA in the bodily, or tissue site in which it is implanted or situated. When the depot resides within a patient's tissue(s), the BA is released in a sustained and controlled manner upon contact with body fluids, water, or other aqueous media primarily by degradation, dissolution, and/or erosion of the crystalline composition, protein gel, or particulate suspension.

### Bone Morphogenetic Proteins

As stated above, BMPs are a preferred exemplary BA for purposes of the present invention. BMPs belong to the TGF-β superfamily. The TGF-β superfamily proteins are cytokines characterized by six-conserved cysteine residues). The human genome contains about 42 open reading frames encoding TGF-β superfamily proteins. The TGF-β superfamily proteins can at least be divided into the BMP subfamily and the TGF-β subfamily based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-β (GDF-6, CDMP-2), BMP-15 (GDF-9), BMP-16, GDF-1, GDF-3, GDF-5 (CDMP-1, MP-52), and GDF-8 (myostatin). For purposes of the present invention, preferred superfamily proteins include BMP-2, -4, -5, -6 and -7 and GDF-5, -6, and -7, as well as MP-52. Particularly preferred proteins include BMP-2, BMP-7 and GDF-5, -6, and -7. A most preferred exemplary BMP is BMP-7. BMPs are also present in other animal species. Furthermore, there is allelic variation in BMP sequences among different members of the human population, and there is species variation among BMPs discovered and characterized to date. As used herein, “BMP subfamily,” “BMPs,” “BMP ligands” and grammatical equivalents thereof refer to the BMP subfamily members, unless specifically indicated otherwise.

The TGF-β subfamily includes, but is not limited to, TGFs (e.g., TGF-β1, TGF-β2, and TGF-β3), activins (e.g., activin A) and inhibins, macrophage inhibitory cytokine-1 (MIC-1), Mullerian inhibiting substance, anti-Mullerian hormone, and glial cell line derived neurotrophic factor (GDNF). As used herein, “TGF-β subfamily,” “TGF-βs,” “TGF-β ligands” and grammatical equivalents thereof refer to the TGF-β subfamily members, unless specifically indicated otherwise.

The TGF-β superfamily is in turn a subset of the cysteine knot Cytokine superfamily. Additional members of the cysteine knot cytokine superfamily include, but are not limited to, platelet derived growth factor (PDGF), vascular endothelial growth factor (VEGF), placenta growth factor (PIGF), noggin, neurotrophins (BDNF, NT3, NT4, and βNGF), gonadotropin, follitropin, lutropin, interleukin-17, and coagulogen.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1990)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-β (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)) The above publications are incorporated herein by reference.

As used herein, “TGF-β superfamily member” or “TGF-β superfamily protein,” means a protein known to those of ordinary skill in the art as a member of the Transforming Growth Factor-β (TGF-β) superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Thus, morphogenic proteins useful in this invention can behave differently in different surroundings. In certain embodiments, a morphogenic protein of this invention can be a homodimer species or a heterodimer species.

The term “osteogenic protein (OP)” refers to a morphogenic protein that is also capable of inducing a progenitor cell to form cartilage and/or bone. The bone can be intramembraneous bone or endochondral bone. Most osteogenic proteins are members of the BMP subfamily and are thus also BMPs. However, the converse can not be true. According to this invention, a BMP identified by DNA sequence homology or amino acid sequence identity must also have demonstrable osteogenic or chondrogenic activity in a functional bioassay to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs may form homodimers. Some BMPs may form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain may noncovalently bind the mature domain and may act as an inhibitor (e.g., Thies et al. (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et. al. (2001) Growth Factors, 18:251-259).

BMP signal transduction is initiated when a BMP dimer binds two type I and two type II serine/threonine kinase receptors. Type I receptors include, but are not limited to, ALK-1, ALK-2 (also called ActRIa or ActRI), ALK-3 (also called BMPRIa), and ALK-6 (also called BMPRIb). Type II receptors include, but are not limited to, ActRIIa (also called ActRII), ActRIIb, and BMPRII. Human genome contains 12 members of the receptor serine/threonine kinase family, including 7 type I and 5 type II receptors, all of which are involved in TGF-β signaling (Manning et al., 2002, the disclosures of which are hereby incorporated by reference). Following BMP binding, the type II receptors phosphorylate the type I receptors, the type I receptors phosphorylate members of the Smad family of transcription factors, and the Smads translocate to the nucleus and activate the expression of a number of genes.

BMPs also interact with inhibitors, soluble receptors, and decoy receptors, including, but not limited to, BAMBI (BMP and activin membrane bound inhibitor), BMPER (BMP-binding endothelial cell precursor-derived regulator), Cerberus, cordin, cordin-like, Dan, Dante, follistatin, follistatin-related protein (FSRP), ectodin, gremlin, noggin, protein related to Dan and cerberus (PRDC), sclerostin, sclerostin-like, and uterine sensitization-associated gene-1 (USAG-1). Furthermore, BMPs may interact with co-receptors, for example BMP-2 and BMP-4 bind the co-receptor DRAGON (Samad et. al. (2005) J. Biol. Chen.), and extracellular matrix components such as heparin sulfate and heparin (Irie et al. (2003) Biochem. Biophys. Res. Commun. 308: 858-865).

As contemplated herein, the term “BMP” refers to a protein belonging to the BMP subfamily of the TGF-β superfamily of proteins defined on the basis of DNA homology and amino acid sequence identity. According to this invention, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7; BMP-2/6; BMP-2/5; BMP-4/7; BMP-4/5; and BMP-4/6 heterodimers. Suitable BMP variants and heterodimers include those set forth in US 2006/0235204; WO 07/087,053; WO 05/097825; WO 00/020607; WO 00/020591; WO 00/020449; WO 05/113585; WO 95/016034 and WO93/009229.

To promote bone growth, the BA of the present invention can be an osteoinductive or osteoconductive substance. Suitable bone growth promoting agents include, for example, a BMP or analogs derived therefrom. The terms “drug,” “medicament,” or “biologic agent”/“BA” (i.e., biologically active agent) as used herein include without limitation biologically, physiologically or pharmacologically active substances that act locally or systemically in the body. A BA is a substance used for the treatment, prevention, diagnosis, cure or mitigation of disease or illness, a substance which affects the structure or function of the body, or pro-drugs, which become biologically active or more active after they have been placed in a predetermined physiological environment. Various forms of the BA can be used which are capable of being released from the crystal, gel, particulate suspension, or pharmaceutical composition into adjacent tissues or fluids. The BAs are water soluble, preferably very slightly water soluble, still more preferably substantially physiologically insoluble, and are diffusible through a carrier, vehicle, or polymeric composition. They can be one or a combination of acidic, basic, or amphoteric salts. They can be one or a combination of nonionic molecules, polar molecules, non-polar molecules, or molecular complexes capable of hydrogen bonding. The BA can be included in the compositions in the form of, for example, an uncharged molecule, a molecular complex, a salt, an ether, an ester, an amide, polymer drug conjugate, or other form to provide the effective biological or physiological activity.

To those skilled in the art, any BA that can be released in an aqueous environment can be utilized in the described pharmaceutical composition. In a preferred embodiment, the BA is proteinaceous. In another preferred embodiment, the BA is minimally soluble. In a more preferred embodiment, the BA is substantially physiologically insoluble. In a further preferred embodiment, the BA is substantially insoluble at physiological pH. In another preferred embodiment, the BA is one that, prepared or manufactured as a crystal, macromolecular gel, or particulate suspension, can persist, after dosing, in vivo, with effective release of active, for 1 hour, more preferably 24 hours, more preferably 48 hours, still more preferably one week, still more preferably one month, yet still more preferably several months. In a particularly preferred embodiment, the BA is prepared or manufactured ex vivo as a crystal, macromolecular gel, or particulate suspension, and only then administered to an individual, thus creating a depot in the individual that can persist, after dosing, in vivo, with effective release of active, for 1 hour, more preferably 24 hours, more preferably 48 hours, still more preferably one week, still more preferably one month, yet still more preferably several months. In a preferred embodiment, the BA is a protein that is substantially physiologically insoluble. In a still more preferred embodiment, the BA is a protein that is substantially insoluble at physiological pH. In another preferred embodiment, the BA is a protein that is conformationally immobile. In a still more preferred embodiment, the BA is a protein that is limited in the conformational movement of its tertiary and/or quaternary structure(s) by covalent bonds. In a preferred embodiment, said covalent bonds are disulfide bridges. In a more particularly preferred embodiment, the BA is a member of the TGF-β superfamily. In a still more particularly preferred embodiment, the BA is selected from the group consisting of BMP-2, BMP-4, BMP-5, BMP-6, BMP-7, GDF-5, GDF-6, GDF-7, as well as any and all variants and homologues thereof. For instance, useful BMPs include those containing sequences, which are homologues or variants, that share at least 50%, preferably at least 60%, more preferably at least 70% and most preferably at least 85%, amino acid sequence identity with the C-terminal cysteine domain of BMP-2, BMP4, BMP-5, BMP-6, BMP-7, GDF-5, GDF-6, or GDF-7. As contemplated herein, preferred BMPs include biologically active variants of any such BMPs, including variants containing conservative amino acid substitutions. All that is required by the present invention is that these variants retain biological activity comparable to the native form. As used herein, the term “BMP related protein” or “BMP related proteins” means any one or all of the foregoing proteins.

Morphogenic proteins useful herein include any known naturally occurring native proteins, including allelic, phylogenetic counterparts and other variants thereof. These variants include forms having varying glycosylation patterns, varying N-termini, and active truncated or mutated forms of a native protein. Useful morphogenic proteins also include those that are biosynthetically produced (e.g., “muteins” or “mutant proteins”) and those that are new, morphogenically active members of the general morphogenic family of proteins.

Also, various forms of a BA can be used. These include without limitation forms such as uncharged molecules, molecular complexes, salts, ethers, esters, amides, etc., which are biologically activated when injected into the body. Preferred BAs include, but are not limited to, proteins having therapeutic or prophylactic activity, including enzymes, growth factors, hormones, differentiation factors, cytokines, chemokines, and antibodies.

### Methods of Treatment

The present invention further provides methods for the treatment of disease, particularly joints impacted by disease, especially osteoarthritis and osteochondral disease. The methods of the present invention comprise the step of administering, or treating an individual with, one or more BA gels, crystals, or particulate suspensions. In a preferred embodiment, the method comprises the steps of administering one or more BA gels, crystals, or particulate suspensions, and also administering one or more additional biologically active agents as disclosed above. In a particularly preferred embodiment, the method comprises the step of administering, or treating an individual with, a BMP gel, crystal, or particulate suspension. In a still more particularly preferred embodiment, the method comprises the step of treating an individual with, or administering, a BMP-7 gel, crystal, or particulate suspension. The methods of the present invention can also comprise the administration, or treatment of an individual with, a pharmaceutical composition comprising a BA gel, crystal, or particulate suspension, and one or more other excipients or agents disclosed herein above including, but not limited to, release modifying agents, plasticizers, carriers, pliability modifiers, tonicity modifiers, co-localized pH modifying agents, or pharmaceutically acceptable solvents and vehicles. The methods of the present invention also include the co-administration to an individual of a pre-precipitated amount of a BA, especially a BMP, with a BA gel, crystal, or particulate suspension. As used herein, “pre-precipitated” refers to a BA that has been precipitated ex vivo prior to administration to an individual, and therefore prior to the creation of an in vivo BA depot within the individual. The methods of the present invention can include administration anywhere in the body, preferably to a skeletal tissue site, preferably to a non-vascularized tissue site, preferably to a non-mineralized skeletal tissue, preferably to the joints, preferably to the inter-articular space, more preferably to the articular cartilage, more preferably to the synovial space, more preferably to the meniscus. The skilled artisan would appreciate that the treatment and administration methods of the present invention can be modified or varied to optimize treatment of an individual in view of numerous factors including, but not limited to, the indication, the pathology of the disease, and the physical characteristics of the individual.

### Therapeutic Interventions

As explained above, the invention also provides methods of treatment by administering a formulation or pharmaceutical composition of the present invention. In the case of any particular BA, the formulations of that BA contemplated herein can be used to treat or prevent any known or potential condition for which the BA is efficacious. For example, the BMP formulations of the invention can be used to treat patients suffering from disease or injury of connective tissues, such as bone and cartilage. Additionally, as described below, the BMP formulations of the invention can be used to treat diseases or injuries of other tissues.

BMPs are capable of inducing the developmental cascade of bone morphogenesis and tissue morphogenesis for a variety of tissues in mammals different from bone or cartilage. This morphogenic activity includes the ability to induce proliferation and differentiation of progenitor cells, and the ability to support and maintain the differentiated phenotype through the progression of events that results in the formation of bone, cartilage, non-mineralized skeletal or connective tissues, and other adult tissues.

For example, BMPs can be used for treatment to prevent loss of and/or increase bone mass in metabolic bone diseases. General methods for treatment to prevent loss of and/or increase bone mass in metabolic bone diseases using osteogenic proteins are disclosed in U.S. Pat. No. 5,674,844, the disclosures of which are hereby incorporated by reference. BMPs of the present invention can be used for periodontal tissue regeneration. General methods for periodontal tissue regeneration using osteogenic proteins are disclosed in U.S. Pat. No. 5,733,878, the disclosures of which are hereby incorporated by reference. BMPs can be used for liver regeneration. General methods for liver regeneration using osteogenic proteins are disclosed in U.S. Pat. No. 5,849,686, the disclosures of which are hereby incorporated by reference. BMPs can be used for treatment of chronic renal failure. General methods for treatment of chronic renal failure using osteogenic proteins are disclosed in U.S. Pat. No. 6,861,404, the disclosures of which are hereby incorporated by reference. BMPs can be used for enhancing functional recovery following central nervous system ischemia or trauma. General methods for enhancing functional recovery following central nervous system ischemia or trauma using osteogenic proteins are disclosed in U.S. Pat. No. 6,407,060, the disclosures of which are hereby incorporated by reference. BMPs can be used for inducing dendritic growth. General methods for inducing dendritic growth using osteogenic proteins are disclosed in U.S. Pat. No. 6,949,505, the disclosures of which are hereby incorporated by reference. BMPs can be used for inducing neural cell adhesion. General methods for inducing neural cell adhesion using osteogenic proteins are disclosed in U.S. Pat. No. 6,800,603, the disclosures of which are hereby incorporated by reference. BMPs can be used for treatment and prevention of Parkinson's disease. General methods for treatment and prevention of Parkinson's disease using osteogenic proteins are disclosed in U.S. Pat. No. 6,506,729, the disclosures of which are hereby incorporated by reference.

Additionally, BMPs can be used to repair diseased or damaged mammalian tissue. The existing tissue at the locus, whether diseased or damaged, provides the appropriate matrix to allow the proliferation and tissue-specific differentiation of progenitor cells. In addition, a damaged or diseased tissue locus, particularly one that has been further assaulted by surgical means, provides a morphogenically permissive environment.

BMPs also can be used to prevent or substantially inhibit scar tissue formation following an injury. It can induce tissue morphogenesis at the locus, preventing the aggregation of migrating fibroblasts into non-differentiated connective tissue. For example, BMPs can be used for protein-induced morphogenesis of substantially injured liver tissue following a partial hepatectomy.

As another example, BMPs can also be used to induce dentinogenesis. To date, the unpredictable response of dental pulp tissue to injury is a basic clinical problem in dentistry. As yet another example, BMPs can induce regenerative effects on central nervous system (CNS) repair can be assessed using a rat brain stab model.

In the case of skeletal disorders, a number of factors can cause or contribute to cartilage degeneration in mammals, including trauma and inflammatory disease. Damage to cells resulting from the effects of inflammatory response has been implicated as the cause of reduced cartilage function or loss of cartilage function in diseases of the joints (e.g., rheumatoid arthritis (RA) and osteoarthritis (OA)). In addition, autoimmune diseases such as systemic lupus erythematosis (SLE) and scleroderma can also be characterized by a degradation of connective tissue. In the case of some cartilage degenerative diseases such as osteoarthritis (OA), the mechanisms that turn the normal aging of articular cartilage into the pathological OA process are currently unknown. Each of the foregoing diseases can be effectively treated with the materials and methods of the present invention.

As stated earlier, the BMP formulations of the invention can be used effectively to treat skeletal diseases or injuries. For example, the formulations can be used to treat a bone fracture, such as an open fracture or a closed fracture. For the treatment of a closed fracture, the formulation is preferably injected at the fracture site. For open fractures, critical size defects or persistent nonunions, the formulations can be administered by surgical implantation at the fracture site. In both cases, the formulation can be administered alone, or in combination with a suitable carrier, matrix or scaffold, such as a bone cement, a calcium phosphate material, a gel material or a collagen matrix. Suitable carriers, matrices and scaffolds include those disclosed in U.S. Pat. Nos. 6,919,308; 6,949,251; and 7,041,641.

In a preferred embodiment, the BMP formulations of the invention can be used to treat a disease or injury resulting in cartilage degradation or a cartilage defect. For example, the formulations can be applied to a cartilage defect site, such as a degenerative intervertebral disc, or other fibrocartilaginous tissue, including a tendon, a ligament or a meniscus. Such methods are set out in U.S. Pat. No. 6,958,149. The formulations of the invention can also be used to treat a defect or degeneration of articular cartilage, as set forth in published PCT application WO 05/115438, such as the cartilage lining of a joint, such as a synovial joint, including a knee, an elbow, a hip, or a shoulder. In this embodiment, the formulation is preferably injected into the synovial space of the joint. In another embodiment, the formulations of the invention are used to treat an articular cartilage defect site, such as a chondral defect or an osteochondral defect, in a joint. Such articular cartilage defects can be the result of a disease process, such as osteoarthritis or rheumatoid arthritis, or due to injury of the joint. In this embodiment, the formulation can be injected into the joint space or it can be surgically implanted. For example, the formulation can be placed within the defect either alone or in combination with one or more additional active agents, a supporting matrix or scaffold, or marrow stromal cells. The formulation can, optionally, be covered with a suitable covering, for example a muscle flap or a bioresorbable membrane, such as a collagen membrane.

### Formulation and Administration

BAs, and especially BMPs, of the present invention can be formulated for administration to a mammal, preferably a human, in need thereof as part of a pharmaceutical composition. The composition can be administered by means including, but not limited to, direct injection or infusion of the crystal, gel, or particulate suspension by syringe. Additionally, the crystal, gel or suspension may be introduced to the tissue by means including, but not limited to, direct surgical implantation, endoscopy, catheterization, or lavage. If applied during surgery, the composition may be flowed onto the tissue, sprayed onto the tissue, painted onto the tissue, or any other means within the skill in the art. Systemic administration of the BA crystal, BA macromolecular gel, and BA particulate suspension compositions of the present invention is also contemplated. In a preferred embodiment, the BA composition is administered subcutaneously. In another preferred embodiment, the BA composition is administered intramuscularly.

The compositions and formulations of the present invention are also amenable to use, implantation, injection, application, or administration in or into both vascularized and non-vascularized tissue sites. In a preferred embodiment, a BA gel, crystal, or particulate suspension is applied, administered, injected, implanted or used in a non-vascularized tissue site. As used herein, “non-vascularized” refers to a tissue or tissue site in which vascularization is minimal or absent. Such non-vascularized tissue sites include, but are not limited to, the joints, preferably the inter-articular space, preferably the meniscus.

The composition may be administered in or with an appropriate carrier or bulking agent including, but not limited to, a biocompatible oil such as sesame oil, hyaluronic acid, cyclodextrins, lactose, raffinose, mannitol, carboxy methyl cellulose, thermo or chemo-responsive gels, sucrose acetate isobutyrate. The skilled artisan would understand that the bulking agent or carrier most amenable to the practice of the present invention would facilitate the delivery of the condensed dosage forms of the BAs disclosed herein wherein the dosage volumes include, but are not limited to, volumes of 20 μl or less. The skilled artisan would also comprehend that the BA macromolecular gels of the present invention can be administered as emulsions or microemulsions. Suspension or bulking media, either water- or oil-based, that are optimal for use with the microemulsions or emulsions as well as the bulking/suspension media optimal for the maintenance of BA crystals can also be easily comprehended by the skilled artisan. In a particularly preferred embodiment of the present invention, a bulking agent can be used in conjunction with a BA of the present invention that is substantially insoluble at physiological pH, to increase the dissolution of the BA crystal or gel such that the bulking agent acts classically as a barrier to release of the BA. In a still more particularly preferred embodiment, the BA is BMP-7. It is within the skill in the art to practice the aforementioned embodiments of the present invention, as well as any and all variants and modifications of the present invention that the skilled artisan would recognize provide sustained, effective post-dosing release of the BA depot in vivo.

Still further, the BMP solid crystals, liquid crystals, macromolecular gels, and particulate suspensions of the present invention can be administered to the mammal in need thereof either alone or in combination with another substance known to have a beneficial effect on tissue morphogenesis. Examples of such substances (herein, cofactors) include without limitation substances that promote tissue repair and regeneration and/or inhibit inflammation. Examples of useful cofactors for stimulating bone tissue growth in osteoporotic individuals, for example, include but are not limited to, vitamin D₃, calcitonin, prostaglandins, parathyroid hormone, dexamethasone, estrogen and IGF-I or IGF-II. Useful cofactors for nerve tissue repair and regeneration can include, but are not limited to, nerve growth factors. Other useful cofactors include symptom-alleviating cofactors, including, but not limited to, antiseptics, antibiotics, antiviral and antifungal agents, analgesics and anesthetics.

As will be appreciated by those skilled in the art, the concentration of the compounds described in a therapeutic composition will vary depending upon a number of factors, including without limitation the dosage of the drug to be administered, the chemical characteristics (e.g., hydrophobicity) of the compounds employed, and the route of administration. The preferred dosage of drug to be administered also is likely to depend on variables including, but not limited to, the type and extent of a disease, tissue loss or defect, the overall health status of the particular patient, the relative biological efficacy of the compound selected, the formulation of the compound, the presence and types of excipients in the formulation, and the route of administration. The therapeutic molecules of the present invention may be provided to an individual where typical doses range from about 10 ng/kg to about 1 g/kg of body weight per day; with a preferred dose range being from about 0.1 mg/kg to 100 mg/kg of body weight, and with a more particularly preferred dosage range of 10-1000 μg/dose. In a particularly preferred embodiment, a dose of 10-1000 μg of a BMP-7 crystal, gel, or particulate suspension is administered to an individual afflicted with osteoarthritis. The skilled clinician would appreciate that the effective doses of the present invention can be modified in light of numerous factors including, but not limited to, the indication, the pathology of the disease, and the physical characteristics of the individual. It is also clearly within the skill in the art to vary, modify, or optimize doses in view of any or all of the aforementioned factors.

Pursuant to the parameters and conditions of the invention, the release of the BA can be controlled. In particular, the rate and extent of release of the BA from an implant, implantable article, device and the like according to the invention can be controlled by variation of the polymer type and molecular weight, use of a rate modifying agent, use of plasticizers and leachable agents and the concentrations and kinds of thermoplastic polymer and BA.

Rate modifying agents, plasticizers and leachable agents can be included to manage the rate of release of BA and the pliability of a matrix in which it is optionally contained. The rate modifying agent can increase or retard the rate of release depending upon the nature of the rate modifying agent incorporated into a matrix. Known plasticizers as well as organic compounds that are suitable for secondary pseudobonding in polymer systems are acceptable as rate modifying agents and also as pliability modifiers and leaching agents. Generally these agents are esters of mono, di and tricarboxylic acids, diols and polyols, polyethers, non-ionic surfactants, fatty acids, fatty acid esters, oils such as vegetable oils, and the like. The concentrations of such agents within the matrix can range in amount up to 60 wt % relative to the total weight of the matrix, preferably up to 30 wt % and more preferably up to 15 wt %. Generally, these rate modifying agents, leaching agents, plasticizers and pliability modifiers and their application are described in U.S. Pat. Nos. 5,702,716 and 5,447,725, the disclosures of which are incorporated herein by reference with the proviso that the polymers to be used are biocompatible and/or biodegradable. The skilled artisan would appreciate that the present invention comprises any and all agents within the art that can increase the solubilization rate of the BA or the degradation rate or erosion rate of any carrier for the BA. Hence, other agents amenable to the practice of the present invention include, but are not limited to, co-localized pH modifying agents and tonicity modifiers. In a particularly preferred embodiment, the composition of the present invention comprises a co-localized pH modifying agent or tonicity modifier provided in a concentration or quantity that substantially increases the solubilization rate of the BA. In another preferred embodiment, the composition of the present invention comprises a co-localized pH modifying agent or tonicity modifier provided in a concentration or quantity that substantially increases the degradation rate or erosion rate of the carrier. The skilled artisan would appreciate that the rate modifying agents, leaching agents, plasticizers, pliability modifiers, pH modifying agents, and tonicity modifiers of the present invention can be substituted, modified, varied in nature or concentration, and optimized in view of numerous factors, including, but not limited to, the desired release rate, the nature of the carrier (if any), the indication, the pathology of the disease, and the physical characteristics of the individual.

Controlled dissolution of the solid or liquid protein crystal, crystal formulation, macromolecular gel or release of the constituent of any formulations can be controlled by numerous factors, including, but not limited to, the surface area of the crystal, particle, or gel; the size of said crystal, particle, or gel; the shape of said crystal, particle or gel; the concentration of any excipient component; the number and nature of any excipient components; the molecular weight of any excipient components; and any combinations of the aforementioned.

Organic solvent, water, or any other fluid may be removed from the crystal by any means including, but not limited to, drying with nitrogen, air or inert gases; vacuum oven drying; lyophilization; washing with a volatile organic solvent followed by evaporation; evaporation in a fume hood; passing a stream of gas over wet crystals, the gas being nitrogen, a Noble gas, carbon dioxide, air, or combinations thereof; or exchange into a biocompatible solvent or aqueous based system for storage and delivery.

Formulations of crystals, gels, or particulate suspensions of this invention can include a combination of the crystal, gel, or suspension and one or more ingredients or excipients, including sugars and biocompatible polymers. Examples of excipients are described in the Handbook of Pharmaceutical Excipients, published jointly by the American Pharmaceutical Association and the Pharmaceutical Society of Great Britain. For the purposes of this application, “formulations” include “crystal formulations.” Furthermore, “formulations” include “protein crystal formulations,” “protein gel formulations,” and “protein suspension formulations.”

As used herein “pharmaceutically effective amount” means an amount of a BA crystal, BA macromolecular gel, or BA particulate suspension that is effective to treat a condition in a living organism to which it is administered over a period of time.

Excipients that may be employed in the making and use of the formulations and pharmaceutical compositions of the present invention include, but are not limited to; acidifying agents, such as, acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid; alcohol denaturants, such as, denatonium benzoate, methyl isobutyl ketone, sucrose octacetate; alkalizing agents, such as, strong ammonia solution, ammonium carbonate, diethanolamine, diisopropanolamine, potassium hydroxide, sodium bicarbonate, sodium borate, sodium carbonate, sodium hydroxide, trolamine; antifoaming agents, such as, dimethicone, simethicone; antimicrobial preservatives, such as, benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol; antioxidants, such as, ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient; buffering agents, such as, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate; chelating agents, such as, edetate disodium, ethylenediaminetetraacetic acid and salts, edetic acid; coating agents, such as, sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein; colors, such as, caramel, red, yellow, black or blends, ferric oxide; complexing agents, such as, ethylenediaminetetraacetic acid and salts (EDTA), edetic acid, gentisic acid ethanolamide, oxyquinoline sulfate; desiccants, such as, calcium chloride, calcium sulfate, silicon dioxide; emulsifying and/or solubilizing agents, such as, acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax; filtering aids, such as, powdered cellulose, purified siliceous earth; glidants and/or anticaking agents, such as, calcium silicate, magnesium silicate, colloidal silicon dioxide, talc; humectants, such as, glycerin, hexylene glycol, propylene glycol, sorbitol; plasticizers, such as, castor oil, diacetylated monoglycerides, diethyl phthalate, glycerin, mono- and di-acetylated monoglycerides, polyethylene glycol, propylene glycol, triacetin, triethyl citrate; polymer membranes, such as, cellulose acetate; solvents, such as, acetone, acetic acid, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water; sorbents, such as, powdered cellulose, charcoal, purified siliceous earth, and carbon dioxide sorbents; stiffening agents, such as, hydrogenated castor oil, cetostearyl alcohol, cetyl alcohol, cetyl esters wax, hard fat, paraffin, polyethylene excipient, stearyl alcohol, emulsifying wax, white wax, yellow wax; suspending and/or viscosity-increasing agents, such as, acacia, agar, alginic acid, aluminum monostearate, bentonite, purified bentonite, magma bentonite, carbomer 934p, carboxymethylcellulose calcium, carboxymethylcellulose sodium, carboxymethylcellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum; and wetting and/or solubilizing agents, such as, benzalkonium chloride, benzethonium chloride, cetylpyridinium chloride, decussate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol.

### Bioactive Co-Agents

The present invention also contemplates “bioactive co-agents” that can be co-administered with the BA crystal, gel, or particulate suspension compositions of the present invention include, but are not limited to, anabolic agents, antacids, anti-asthmatic agents, anti-cholesterolemic and anti-lipid agents, anti-coagulants, anti-convulsants, anti-diarrheals, anti-emetics, anti-infective agents including, for example, antibacterial and antimicrobial agents, anti-inflammatory agents, anti-manic agents, antimetabolite agents, anti-nauseants, anti-neoplastic agents, anti-bone resorption agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

More particularly, the bioactive co-agents preferred for co-administration with the crystals, gels, or particulate suspensions of the present invention include, but are not limited to, androgen inhibitors, polysaccharides, growth factors, hormones, bisphosphonates, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines. Other representative bioactive co-agents that can be co-administered with the crystalline, gel, and particulate suspension compositions of the present invention include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, .beta.-adrenergic blocking agents, nutritional agents, and the benzophenanthridine alkaloids. The bioactive co-agent may further be a substance capable of acting as a stimulant, sedative, hypnotic, analgesic, anticonvulsant, and the like.

The bioactive co-agent may also be a substance, or metabolic precursor thereof, which is capable of promoting growth and survival of cells and tissues, or augmenting the activity of functioning cells, as for example, blood cells, neurons, muscle, bone marrow, bone cells and tissues, and the like. For example, bioactive co-agents that may be co-administered with the crystalline, gel, or particulate suspension compositions of the present invention may include without limitation a nerve growth promoting substance, as for example, a ganglioside, phosphatidylserine, a nerve growth factor, brain-derived neurotrophic factor. The bioactive co-agent may also be a growth factor for soft or fibrous connective tissue as, for example, a fibroblast growth factor, an epidermal growth factor, an endothelial cell growth factor, a platelet derived growth factor, an insulin-like growth factor, a periodontal ligament cell growth factor, to name but a few.

### Crystallinity

The crystallization of macromolecules, including proteins, can greatly aid in their storage, as well as their in vivo delivery. However, stability of these crystals can present numerous problems, since there are very few methods for preparing large quantities of macromolecule crystals that are stable outside of the mother liquor. In particular, protein crystals must be handled with greater care since they are extremely fragile and contain a good deal of solvent. One technique commonly employed allows for the separation of the crystal from the mother liquor and its insertion into a capillary tube with subsequent air-tight sealing of the tube using, for instance, dental wax or silicone grease, along with a small amount of the mother liquor to maintain the crystal's hydration. (McPherson, A., Preparation and Analysis of Protein Crystals, Robert E. Krieger Publishing, Malabar, p. 214 (1989)). Macromolecular crystals can also be maintained at cryogenic temperatures using methods well known in the art. Preparation of the crystal with subsequent rapid cooling can prevent the formation of ice lattices within the aqueous medium. In lieu of the ice that would normally form, a rigid glass forms instead, encasing the crystal without damaging it. The resulting crystals are stored at 100K to prevent disintegration of the crystal. (Rodgers, D. W., in Methods in Enzymology (Eds., Carter, C. W. and Sweet, R. M.) Academic Press, v. 276, p. 183 (1997)). Although this technique allows storage of crystals outside of the mother liquor, it requires maintenance of the crystal at temperatures at or below 100K.

Dried crystals can also be prepared by lyophilization, a technique that requires rapid cooling of the material. This limits the application of the technique to products that are stable under such frozen conditions. The technique requires that the aqueous solution is frozen first at a temperature of between −40 and −50 degrees Celsius. The resulting ice is then removed under vacuum, since ice formulation can potentially destroy the protein crystal lattice.

Optimally, crystalline macromolecules should be stable at ambient temperatures for convenient storage. Crystalline macromolecules, particularly crystalline proteins, are particularly advantageous for use as therapeutics and vaccines. The present invention provides formulations and compositions of crystalline BAs, particularly crystalline proteins, even more particularly crystalline BMPs, that are solid particles or dispersed in a non-aqueous solvent. In an embodiment of the present invention, the BA compositions of the present invention comprise, in place of the mother liquor, a non-aqueous solvent. In another embodiment of the present invention, a slurry of the crystalline BA can be rendered solid by spinning out the first solvent and washing the remaining crystalline BA solid using a second organic solvent to remove water with subsequent evaporation of the non-aqueous solvent.

To optimize the preparation and maintenance of protein crystals, it is possible to leave the crystals in the mother liquor during the course of the protein crystal production process, potentially encapsulated in polymeric carriers. Polymer processing conditions are compatible with the many of the compounds used in protein crystallization including, but not limited to, salts, PEG, and organic solvents. The skilled artisan would also appreciate that crystal dissolution within the mother liquor can be controlled by conditions including, but not limited to, pH; temperature; presence of metal ions, such as Zn, Cu and Ca; and the concentration of precipitants. The skilled artisan would also recognize that, by varying these conditions, one can slow down the dissolution of crystals for several hours. The skilled artisan would further appreciate that the process of microparticulate formation is very fast and normally takes seconds to minutes to complete. Furthermore, filtration can be used to remove the mother liquor, leaving a crystalline paste that can be dried by air, under vacuum, washing with miscible organic solvents, and/or by lyophilization, leaving dried crystals. The skilled artisan would also appreciate that crystals, including protein crystals, can be chemically crosslinked to greatly reduce, or eliminate altogether, the propensity to dissolve in aqueous, or even non-aqueous, media. It is also within the art to manipulate or control the crystal size or shape during the crystallization process, resulting in a range of crystal morphologies with differing dissolution kinetics and, therefore, differing sustained release profiles compared to amorphous proteins.

In another embodiment of this invention, an excipient is dissolved in a solution other than the mother liquor, and the BA crystals are removed from the mother liquor and suspended in the excipient solution.

The skilled artisan would also appreciate that macromolecules, such as BAs, are easier to crystallize, and have more stable resulting crystals and gels, if the macromolecules have low solubility and have tertiary and/or quaternary structures that are relatively conformationally immobile. In particular, proteins that have strong interactions, including, but not limited to, covalent bonds between tertiary structures or between polypeptides in a multimer, for instance, have fewer conformational degrees of freedom than proteins lacking such interactions. The decreased conformational mobility makes the proteins more amenable to the local ordering that may aid crystallization and gel-formation. Furthermore, proteins with low solubility also tend to aggregate, their hydrophobic surfaces forming, for instance, extensive Van der Waals contacts that encourage local ordering of the proteins which in turn may aid in crystallization and gel-formation. The skilled artisan would appreciate that the proteins of the TGF-β superfamily and especially the BMPs are, relative to other proteins, conformationally immobile and substantially physiologically insoluble, and are therefore particularly amenable to the making and use of the crystals, gels, and particulate suspensions of the present invention. The skilled artisan would appreciate that varying degrees of solubility and conformational immobility can alter the nature and morphology of crystals and it is also within the art for the routineer to modify and vary the conditions under which such proteins optimally crystallize.

The possible advantage of the crystalline form as opposed to a pre-precipitated form is the reduced surface area to volume ratio which can increase sustained release levels. The crystalline form, with its reduced surface area to volume ratio, is also likely less irritating to tissues at the site of administration since the lower surface area per given dose mitigates or reduces the local irritation from precipitation. In a preferred embodiment, the BA crystals can be administered using a syringe with a gauge between 12 and 30. In a still more particularly preferred embodiment, the BA crystals can be administered using a syringe with a gauge between 16 and 26. The skilled artisan would appreciate that the manipulation of the surface area/volume ratio of the BA crystals and gels of the present invention can modify the dissolution/release rate according to her desires with such manipulation well within the skill in the art.

The present invention also envisions the practice of all means known and commonly used in the art for crystallizing proteins including, but not limited to, concentration-through-evaporation, sublimation, diffusion gradient techniques, and batch techniques.

### Protein Gels

Protein gels of the present invention can be achieved with BAs, and especially BMPs, of varying protein concentrations and in a variety of different buffers known to the skilled artisan, through techniques including, but not limited to, centrifugation, evaporation, solvent exchange, tangential flow filtration, and dialysis. “Protein gel” as used herein does not refer to the use of carrier gels, such as PLG-PEG, or similar polymer compositions. Rather, “protein gel” refers to a state of gelation and/or a gelation phenomenon attributable to the macromolecular ordering of the proteinaceous BA per se. The skilled artisan would understand that the present invention includes any and all techniques commonly in use for procuring protein gels and is thus enabled by the techniques known in the art to practice any and all protein gels of the present invention.

A possible advantage of the gel form as opposed to a pre-precipitated form is a reduced surface area to volume ratio which can increase sustained release levels. A gel form, with its reduced surface area to volume ratio, is also less irritating to tissues at the site of administration since the lower surface area per given dose mitigates or reduces the local irritation from precipitation. In a preferred embodiment, the gels of the present invention consist of a BA and a solvent. In a preferred embodiment, the protein gels of the present invention consist of protein and a solvent. An exemplary protein gel of a preferred protein, BMP-7, is set forth in Example 2.

### Particulate Suspensions

Particulate suspensions of the present invention can be achieved with BAs, especially BMPs, of varying protein concentrations and in a variety of different buffers known to the skilled artisan including, but not limited to, water and phosphate buffered saline (PBS). The skilled artisan would understand that the present invention includes any and all techniques commonly in use for procuring stable particulate suspensions and is thus enabled by the techniques known in the art to practice any and all particulate suspensions of the present invention.

Gel suspensions and crystal suspensions are contemplated, both alone and in combination with a suspending vehicle. Suspending vehicles of the present invention include both aqueous and non-aqueous vehicles. The aqueous solvents contemplated by the present invention include, but are not limited to, saline, carboxymethylcellulose (CMC), and hyaluronic acid. The non-aqueous vehicles contemplated by the present invention include, but are not limited to, sesame oil. Contemplated suspensions also include, but are not limited to, precipitated and pre-precipitated BAs. In a preferred embodiment, the precipitated or pre-precipitated BA is a protein that may be, by way of illustration only, lyophilized cake.

### Pharmaceutical Compositions

The present invention also provides pharmaceutical compositions useful for the treatment of disease, particularly joints impacted by disease, especially osteoarthritis and osteochondral disease. The pharmaceutical compositions of the present invention comprise one or more BA gels, crystals, or particulate suspensions and a pharmaceutically acceptable solvent, vehicle, or carrier. In a preferred embodiment, the pharmaceutical compositions of the present invention comprise one or more BA gels, crystals, or particulate suspensions, and one or more additional biologically active agents. In a particularly preferred embodiment, the BA is a BMP. In a still more particularly preferred embodiment, the BA is BMP-7. The pharmaceutical compositions of the present invention can also comprise one or more other excipients or agents disclosed herein above including, but not limited to, release modifying agents, plasticizers, carriers, pliability modifiers, tonicity modifiers, or co-localized pH modifying agents. The skilled artisan would appreciate that the pharmaceutical compositions of the present invention can be modified or varied to optimize treatment of an individual in view of numerous factors including, but not limited to, the indication, the pathology of the disease, and the physical characteristics of the individual.

### Kits

The present invention also provides kits useful for the treatment of disease, particularly joints impacted by disease, especially osteoarthritis and osteochondral disease. The kits of the present invention comprise one or more BA gels, crystals, or particulate suspensions. In a preferred embodiment, the kits of the present invention comprise one or more BA gels, crystals, or particulate suspensions, and one or more additional biologically active agents. In a particularly preferred embodiment, the BA is a BMP. In a still more particularly preferred embodiment, the BA is BMP-7. The kits of the present invention can also comprise one or more other excipients or agents disclosed herein above including, but not limited to, release modifying agents, plasticizers, carriers, pliability modifiers, tonicity modifiers, co-localized pH modifying agents, or pharmaceutically acceptable solvents and vehicles. The skilled artisan would appreciate that the kits of the present invention can be modified or varied to optimize treatment of an individual in view of numerous factors including, but not limited to, the indication, the pathology of the disease, and the physical characteristics of the individual.

### EXAMPLES

### 1. Crystals and Protein Kinetics Modeling

BMP-7 crystals were grown by vapor diffusion methods in a sitting drop tray at 19 degrees C. One well contained multiple crystals at approximately 0.1 mm size which were produced using 7.7 mg/mL of BMP-7, with a well solution of 16% 2-methyl-2,4,-pentandiol (MPD) and 135 mM sodium citrate (pH 4.8).

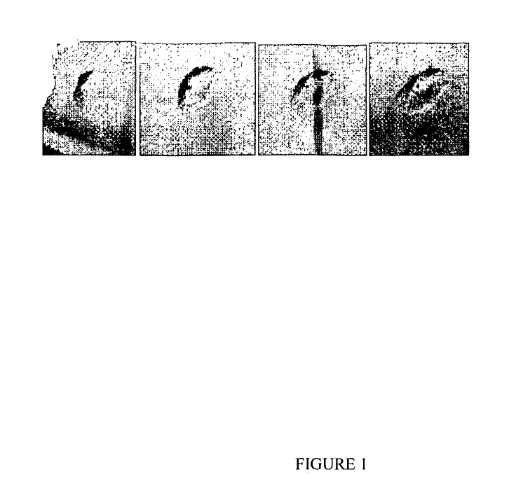

In a sitting drop crystallization tray, 35 microliters of test solution was placed into the post. A crystal was manually transferred using a loop into teach of three solutions: 50 mM acetic acid, phosphate buffered saline (PBS), and bovine synovial fluid. The crystals were observed by a stereo microscope and photographed at 1, 5, 22, and 96 hours with storage under ambient room temperature (approximately 19 degrees C.) in each of the three solutions (FIGS. 1-3).

The crystal that was transferred into 50 mM acetic acid was the least stable (FIG. 1). The edges were observed to have slightly dissolved within the first hour of transfer. Further degradation of the crystal was observed with prolonged exposure.

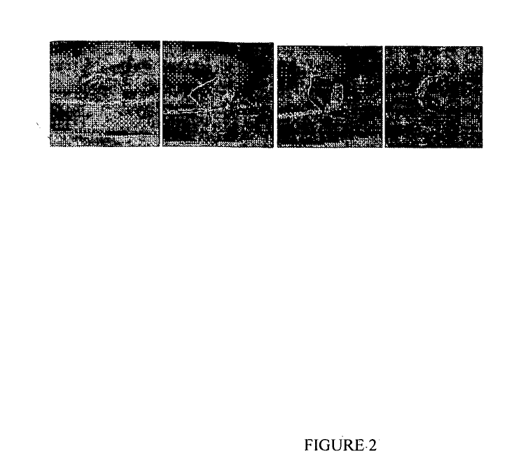

When the crystal was transferred into PBS, a few cracks were produced in the crystal during the initial equilibration (FIG. 2). Prolonged storage in PBS did not result in significant observable changes in the crystal.

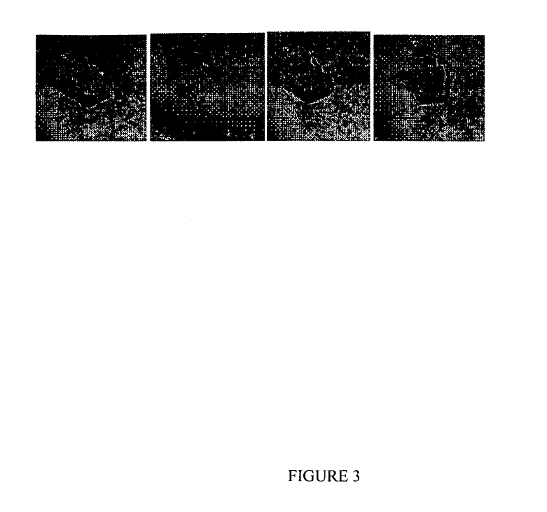

When a crystal was transferred into bovine synovial fluid, some internal cracking was observed (FIG. 3). Further equilibration in the synovial fluid did not appear to alter the edges of the crystal.

These results indicate such a crystal would provide a sustained release depot in the knee to stimulate cartilage repair, for instance. The size of the crystal (greater than the MW cut off of the synovial membrane) helps retain the material in the knee, and provides prolonged delivery time for the protein due to slow dissolution.

The release profile of the BMP crystals may be manipulated to give desired release kinetics. For instance, by injecting a pre-precipitated dose like BMP-7 crystals or a lyophilized BMP-7 protein suspended in saline higher sustained release levels may be reached and a lower Cₘₐₓ level may be achieved. Furthermore, the release rate may be regulated by local injection of solubilized protein, i.e., suspended in saline, thus shifting the release equilibrium. This can take the form of either co-administration with the crystal or protein gel, or can take place as a secondary administration after the initial administration of the crystal or the protein gel.

### 2. High Concentration Protein Gels

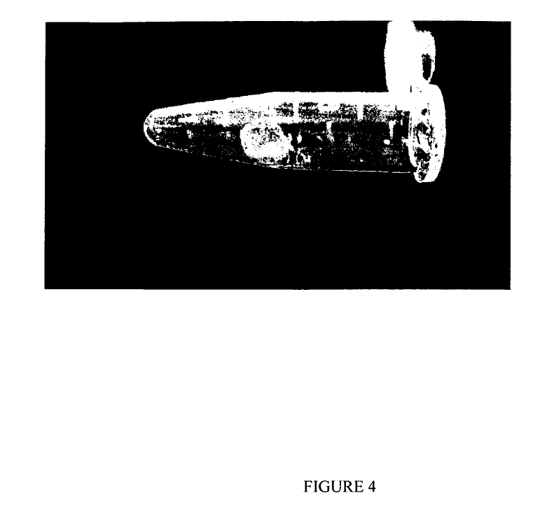

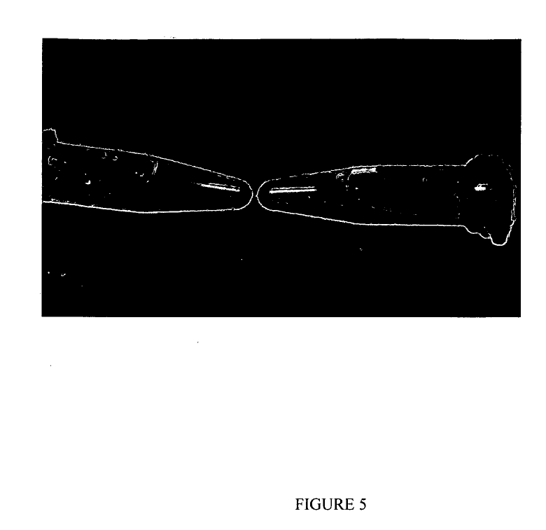

A high concentration protein gel (HCPG) comprising BMP-7 was prepared by centrifugal concentration of BMP-7 in 50 mM acetic acid (approximately 40 mg/ml). (see FIG. 4, the BMP-7 HCPG at T=0) It was observed that such gels show a precipitation halo on the exterior of the gels that over 24 hours extended into the interior of the gel, but not in a complete manner. (see FIG. 5) The HCPG provides a readily manufactured self depot with a solubilization front with at least a 10 times greater concentration of BMP-7 than the equivalent amount in a 1 mg bolus administered directly to the site of interest.

### EQUIVALENTS

The invention may be embodied in other specific forms without departing from the spirit or essential characteristics thereof. The present embodiments are therefore to be considered illustrative and not restrictive, the scope of the invention being indicated by the appended claims rather than by the foregoing description, and all changes which come within the meaning and range of equivalency of the claims are therefore intended to be embraced therein.

1. A composition suitable for implantation at a tissue site, the composition comprising a biologic agent wherein said biologic agent is selected from the group consisting of a crystal, a macromolecular gel or a particulate suspension and further wherein said biologic agent is released in a sustained-release manner at the tissue site in an amount effective to ameliorate an injury or disease at the tissue site.

2. The composition of claim 1, wherein the biologic agent is proteinaceous.

3. The composition of claim 2, wherein the biologic agent is a minimally soluble protein.

4. The composition of claim 3, wherein the biologic agent is substantially insoluble at physiological pH.

5. The composition of claim 4, wherein the biologic agent is a member of the TGF-beta superfamily of proteins.

6. The composition of claim 5, wherein the biologic agent is selected from the group consisting of BMP-2 (SEQ ID NO:1), BMP-4 (SEQ ID NO:3), BMP-5 (SEQ ID NO:7), BMP-6 (SEQ ID NO:9), BMP-7 (SEQ ID NO:11), GDF-5 (SEQ ID NO:13), GDF-6 (SEQ ID NO:15) and GDF-7 (SEQ ID NO:17), and sequence variants of any one of the foregoing.

7. The composition of claim 5, wherein the biologic agent is selected from the group consisting of BMP-2 (SEQ ID NO:1), BMP-7 (SEQ ID NO:11), GDF-5 (SEQ ID NO:13), GDF-6 (SEQ ID NO:15) and GDF-7 (SEQ ID NO:17).

8. The composition of claim 5, wherein the biologic agent is selected from the group consisting of GDF-5 (SEQ ID NO:13), GDF-6 (SEQ ID NO:15) and GDF-7 (SEQ ID NO:17).

9. The composition of claim 5, wherein the biologic agent is BMP-7 (SEQ ID NO:11).

10. The composition of claim 5, wherein the biologic agent is a member of the BMP subfamily of the TGF-beta superfamily of proteins.

11. The composition of claim 10, wherein the biologic agent is a protein having at least about 50% amino acid sequence identity with a member of the BMP subfamily within the conserved C-terminal cysteine-rich domain.

12. The composition of claim 4, wherein the biologic agent is a protein which is not a member of the TGF-beta superfamily of proteins.

13. The composition of claim 1, wherein the biologic agent is a solid or liquid crystal and the tissue site is vascularized or non-vascularized.

14. The composition of claim 1, wherein the biologic agent is a macromolecular gel and the tissue site is vascularized or non-vascularized.

15. The composition of claim 1, wherein the biologic agent is a particulate suspension and the tissue site is vascularized or non-vascularized.

16. The composition of claim 13, H, or 15, wherein the tissue site is a joint.

17. The composition of claim 13, 14, 15, or 16, wherein the tissue site is the inter-articular space.

18. The composition of claim 17, wherein the biologic agent is BMP-7 (SEQ ID NO:11).

19. The composition of claim 1, wherein the crystal, macromolecular gel or particulate suspension are formed ex vivo.

20. The composition of claim 1, further comprising a release modifying agent.

21. The composition of claim 1, further comprising a bulking agent.

22. The composition of claim 1, wherein the composition is in an amount effective to ameliorate skeletal tissue injury or disease selected from the group consisting of metabolic bone disease, osteoarthritis, osteochondral disease, rheumatoid arthritis, osteoporosis, Paget's disease, periodontitis, and dentinogenesis.

23. The composition of claim 1, wherein the composition is in an amount effective to ameliorate non-mineralized skeletal tissue injury or disease selected from the group consisting of osteoarthritis, osteochondral disease, chondral disease, rheumatoid arthritis, trauma-induced and inflammation-induced cartilage degeneration, age-related cartilage degeneration, articular cartilage injuries and diseases, full thickness cartilage defects, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, and injuries and diseases of ligament, tendon, synovial capsule, synovial membrane and meniscal tissues.

24. The composition of claim 23, wherein the composition is in an amount effective to ameliorate tissue injury selected from the group consisting of: trauma-induced and inflammation-induced cartilage degeneration, articular cartilage injuries, full thickness cartilage defects, superficial cartilage defects, herniation and rupture of intervertebral discs, degeneration of intervertebral discs due to an injury(s), and injuries of ligament, tendon, synovial capsule, synovial membrane and meniscal tissues.

25. The composition of claim 22, wherein the disease is osteoarthritis or an osteochondral disease.

26. The composition of claim 1, wherein the composition is in an amount effective to ameliorate injury or disease of a tissue selected from the group consisting of liver disease, liver ressection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, and ulcerative diseases of the gastrointestinal tract.

27. A method of treatment of an injured or diseased tissue, the method comprising the step of: providing to a tissue site a composition suitable for implantation at, adjacent or in the vicinity of an injured or diseased tissue wherein the composition comprises a biologic agent selected from the group consisting of a crystal, a macromolecular gel or a particulate suspension, and further wherein said biologic agent is released in a sustained-release manner at the tissue site in an amount effective to treat the injured or diseased tissue.

28. The method of claim 27, wherein the biologic agent is a crystal, macromolecular gel or particulate suspension of BMP-7 (SEQ ID NO:11).

29. The method of claim 27, wherein the biologic agent is a solid or liquid crystal.

30. The method of claim 27, wherein the injured or diseased tissue is a non-vascularized tissue.

31. The method of claim 27, wherein the tissue site of implantation is inter-articular.

32. The method of claim 27, wherein the diseased tissue results from osteoarthritis or osteochondral disease.

33. The method of claim 27, wherein said biologic agent is released in a sustained release manner for at least about 2-7 days.

34. The method of claim 31, wherein said effective amount for treatment of osteoarthritis is about 10 to about 1000 micrograms.

35. A pharmaceutical composition for treatment of an injured or diseased tissue comprising the composition of claim 1 and a pharmaceutically-acceptable vehicle.

36. A kit comprising the composition of claim 1.

37. A composition suitable for systemic administration, the composition comprising a biologic agent wherein said biologic agent is selected from the group consisting of a crystal, a macromolecular gel, or a particulate suspension and further wherein said biologic agent is released in a timed-release manner in an amount effective to ameliorate an injury or disease.

38. The composition of claim 37, wherein systemic administration is either subcutaneous or intramuscular.


SEQUENCE LISTING (18 sequences)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - DNA, 3150 bp, Homo sapiens
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtc

SEQ ID NO: 3 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 4 - DNA, 1957 bp, Homo sapiens
aagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaaaaaaaaaaaaaaaaa

SEQ ID NO: 5 - DNA, 1748 bp, Homo sapiens
aagaggaggaaggaagatgcgagaaggcagaggaggagggagggagggaaggagcgcggagcccggcccggaagctaggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaaaaaaaaaaaaaaaaa

SEQ ID NO: 6 - DNA, 1802 bp, Homo sapiens
gaaggaagtggcgggggaaggagtgtggtggtggtttaaaaaataagggaagccgaggcgagagagacgcagacgcagaggtcgagcgcaggccgaaagctgttcaccgttttctcgactccggggaacatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaaaaaaaaaaaaaaaaa

SEQ ID NO: 7 - protein, 408 aa, Homo sapiens; identical to SEQ ID NO: 3

SEQ ID NO: 8 - DNA, 2207 bp, Homo sapiens
ctcttgaagagggctggtatatttgtgcctgctggaggtggaattaacagtaagaaggagaaagggattgaatggacttacaggaaggatttcaagtaaattcagggaaacacatttacttgaatagtacaacctagagtattattttacactaagacgacacaaaagatgttaaagttatcaccaagctgccggacagatatatattccaacaccaaggtgcagatcagcatagatctgtgattcagaaatcaggatttgttttggaaagagctcaagggttgagaagaactcaaaagcaagtgaagattactttgggaactacagtttatcagaagatcaacttttgctaattcaaataccaaaggcctgattatcataaattcatataggaatgcataggtcatctgatcaaataatattagccgtcttctgctacatcaatgcagcaaaaactcttaacaactgtggataattggaaatctgagtttcagctttcttagaaataactactcttgacatattccaaaatatttaaaataggacaggaaaatcggtgaggatgttgtgctcagaaatgtcactgtcatgaaaaataggtaaatttgttttttcagctactgggaaactgtacctcctagaaccttaggttttttttttttttaagaggacaagaaggactaaaaatatcaacttttgcttttggacaaaaatgcatctgactgtatttttacttaagggtattgtgggtttcctctggagctgctgggttctagtgggttatgcaaaaggaggtttgggagacaatcatgttcactccagttttatttatagaagactacggaaccacgaaagacgggaaatacaaagggaaattctctctatcttgggtttgcctcacagacccagaccattttcacctggaaaacaagcgtcctctgcacctctctttatgctggatctctacaatgccatgaccaatgaagaaaatcctgaagagtcggagtactcagtaagggcatccttggcagaagagaccagaggggcaagaaagggatacccagcctctcccaatgggtatcctcgtcgcatacagttatctcggacgactcctctgaccacccagagtcctcctctagccagcctccatgataccaactttctgaatgatgctgacatggtcatgagctttgtcaacttagttgaaagagacaaggatttttctcaccagcgaaggcattacaaagaatttcgatttgatcttacccaaattcctcatggagaggcagtgacagcagctgaattccggatatacaaggaccggagcaacaaccgatttgaaaatgaaacaattaagattagcatatatcaaatcatcaaggaatacacaaatagggatgcagatctgttcttgttagacacaagaaaggcccaagctttagatgtgggttggcttgtctttgatatcactgtgaccagcaatcattgggtgattaatccccagaataatttgggcttacagctctgtgcagaaacaggggatggacgcagtatcaacgtaaaatctgctggtcttgtgggaagacagggacctcagtcaaaacaaccattcatggtggccttcttcaaggcgagtgaggtacttcttcgatccgtgagagcagccaacaaacgaaaaaatcaaaaccgcaataaatccagctctcatcaggactcctccagaatgtccagtgttggagattataacacaagtgagcaaaaacaagcctgtaagaagcacgaactctatgtgagcttccgggatctgggatggcaggactggattatagcaccagaaggatacgctgcattttattgtgatggagaatgttcttttccacttaacgcccatatgaatgccaccaaccacgctatagttcagactctggttcatctgatgtttcctgaccacgtaccaaagccttgttgtgctccaaccaaattaaatgccatctctgttctgtactttgatgacagctccaatgtcattttgaaaaaatatagaaatatggtagtacgctcatgtggctgccactaatattaaataatattgataataacaaaaagatctgtattaaggtttatggctgcaataaaaagcatactttcagacaaacggggaatttcctaaaaaaaaaaaaaaaaaaaaaaaaaaaaaaaaaaaaaaa

SEQ ID NO: 9 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 10 - DNA, 3105 bp, Homo sapiens
caactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcaatgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttcccttgtagaaagtgaggctcagattaaattttagaatattttctaaatgtctttttcacaatcatgtactgggaaggcaatttcatactaaactgattaaataatacatttataatctacaactgtttgcacttacagctttttttgtaaatataaactataatttattgtctattttatatctgttttgctgtaacattgaaggaaagaccagacttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggca

SEQ ID NO: 11 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 12 - DNA, 1896 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 13 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 14 - DNA, 2383 bp, Homo sapiens
ctccttcaagccctcagtcagttgtgcaggagaaagggggcggttggctttctcctttcaagaacgagttattttcagctgctgactggagacggtgcacgtctggatacgagagcatttccactatgggactggatacaaacacacacccggcagacttcaagagtctcagactgaggagaaagcctttccttctgctgctactgctgctgccgctgcttttgaaagtccactcctttcatggtttttcctgccaaaccagaggcacctttgctgctgccgctgttctctttggtgtcattcagcggctggccagaggatgagactccccaaactcctcactttcttgctttggtacctggcttggctggacctggaattcatctgcactgtgttgggtgcccctgacttgggccagagaccccaggggaccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggcccccggaggcgggcgggctgcccagctgaagctgtccagctgccccagcggccggcagccggcctccttgctggatgtgcgctccgtgccaggcctggacggatctggctgggaggtgttcgacatctggaagctcttccgaaactttaagaactcggcccagctgtgcctggagctggaggcctgggaacggggcagggccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccactggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcaggtagcagcactggccctctgtcttcctgggtggcacatcccaagagccccttcctgcactcctggaatcacagaggggtcaggaagctgtggcaggagcatctacacagcttgggtgaaaggggattccaataagcttgctcgctctctgagtgtgacttgggctaaaggcccccttttatccacaagttcccctggctgaggattgctgcccgtctgctgatgtgaccagtggcaggcacaggtccagggagacagactctgaatgggactgagtcccaggaaacagtgctttccgatgagactcagcccaccatttctcctcacctgggccttctcagcctctggactctcctaagcacctctcaggagagccacaggtgccactgcctcctcaaatcacatttgtgcctggtgacttcctgtccctgggacagttgagaagctgactgggcaagagtgggagagaagaggagagggcttggatagagttgaggagtgtgaggctgttagactgttagatttaaatgtatattgatgagataaaaagcaaaactgtgcct

SEQ ID NO: 15 - protein, 455 aa, Homo sapiens
MDTPRVLLSAVFLISFLWDLPGFQQASISSSSSSAELGSTKGMRSRKEGKMQRAPRDSDAGREGQEPQPRPQDEPRAQQPRAQEPPGRGPRVVPHEYMLSIYRTYSIAEKLGINASFFQSSKSANTITSFVDRGLDDLSHTPLRRQKYLFDVSMLSDKEELVGAELRLFRQAPSAPWGPPAGPLHVQLFPCLSPLLLDARTLDPQGAPPAGWEVFDVWQGLRHQPWKQLCLELRAAWGELDAGEAEARARGPQQPPPPDLRSLGFGRRVRPPQERALLVVFTRSQRKNLFAEMREQLGSAEAAGPGAGAEGSWPPPSGAPDARPWLPSPGRRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 16 - DNA, 3716 bp, Homo sapiens
cccgaggagccgggccccggccgctgtccagccgctccgtgccccgcgcgtcctgcgccgccgccaccgcctcctggggagacgcagccacttgcccgccatggatactcccagggtcctgctctcggccgtcttcctcatcagttttctgtgggatttgcccggtttccagcaggcttccatctcatcctcctcgtcgtccgccgagctgggttccaccaagggcatgcgaagccgcaaggaaggcaagatgcagcgggcgccgcgcgacagtgacgcgggccgggagggccaggaaccacagccgcggcctcaggacgaaccccgggctcagcagccccgggcgcaggagccgccaggcaggggtccgcgcgtggtgccccacgagtacatgctgtcaatctacaggacttactccatcgctgagaagctgggcatcaatgccagctttttccagtcttccaagtcggctaatacgatcaccagctttgtagacaggggactagacgatctctcgcacactcctctccggagacagaagtatttgtttgatgtgtccatgctctcagacaaagaagagctggtgggcgcggagctgcggctctttcgccaggcgccctcagcgccctgggggccaccagccgggccgctccacgtgcagctcttcccttgcctttcgcccctactgctggacgcgcggaccctggacccgcagggggcgccgccggccggctgggaagtcttcgacgtgtggcagggcctgcgccaccagccctggaagcagctgtgcttggagctgcgggccgcatggggcgagctggacgccggggaggccgaggcgcgcgcgcggggaccccagcaaccgccgcccccggacctgcggagtctgggcttcggccggagggtgcggcctccccaggagcgggccctgctggtggtattcaccagatcccagcgcaagaacctgttcgcagagatgcgcgagcagctgggctcggccgaggctgcgggcccgggcgcgggcgccgaggggtcgtggccgccgccgtcgggcgccccggatgccaggccttggctgccctcgcccggccgccggcggcggcgcacggccttcgccagtcgccatggcaagcggcacggcaagaagtccaggctacgctgcagcaagaagcccctgcacgtgaacttcaaggagctgggctgggacgactggattatcgcgcccctggagtacgaggcctatcactgcgagggtgtatgcgacttcccgctgcgctcgcacctggagcccaccaaccacgccatcatccagacgctgatgaactccatggaccccggctccaccccgcccagctgctgcgtgcccaccaaattgactcccatcagcattctatacatcgacgcgggcaataatgtggtctacaagcagtacgaggacatggtggtggagtcgtgcggctgcaggtagcggtgcctttcccgccgccttggcccggaaccaaggtgggccaaggtccgccttgcaggggaggcctggctgcagagaggcggaggaggaagctggcgctgggggaggctgagggtgagggaacagcctggatgtgagagccggtgggagagaagggagcgcagccttcccagtaacttctacctgccagcccagagggaaatatggattttcacaccttgcctggccaccctggaaaaacaagccaaggaggatttcttttgttctgttttctctctctctctctctctctctctctctctctctctctctctctctattactgtggctttggatttccttatgtgtcttacaggctttgatagaaggggaggggaggagagatgcatacccgtttctcaactgctccatggattgaaaaaataacagtttaaaaagggaaacaatgtgggaggaagaatcaccgttgacgcatcttgatttggttggtttttacatgtgtaaagaaggtggggtctctggccatgtcatagcccatgtcttgtgccctcccacacagaaagtgttagatagggaaattggcaaaaagaatagttaagtcaggaatggtcctgcctatagaagagctttgagagaggtgggcccacgggtgcccctctcacccatttgtgtactctgtgagtttaccagctctgccctggcctctttcggtaccaggaactggcaaccttcatctcactcctgagggcccaggtctctgccttcattgttgctttttctggtgggggcaaggggagctggtatggatggaatgacaagaattagtccaaatggaaccccttgaaggataatgagaaaccacaaggcctgcctctgactggggctgacacggaggtgcattagcccaggctggaggtagcccacccaaatgccctttctgattctaattgatttctttcaacagaatttgccaaaattcagacatgcacttctaaggggaaggtgattttccagttcaaaaaaatgggcaggagtggggaacaaaacaattaacgtaagagctacaaaggagggaaaaggaaccaagaagtagaaggagtcccatcaggagggaagatggtgggcctcagggaggatggggatcaagggacaggccaggagccaggagtggggaagggagggatgaaaggggacacaagtccctgtctctgaagtttctttaaaatctgagttccctcccctctctttgacattcctgaaagattaccagccagcaatagcccagggctcccccaaaagaattggttcagattgtaattatcagttaggcaatgtttttaaaacttagtaatgagaaactgtgaaaagagccaagtgttacattgagcttggggtgggagatggggaacaggcagtgaggaaggagacaggggtggaattcgtcttctgggaggaagctggagagagcacagtgaaattgaaatacccattcccagatagtcaaaaacatgaactttcccccagcctgcaccagtattgttttcaaacattgcccatgagtaggccctttgaagagttagcttcctcctcatctttgactataaaattgtttaatcaatggaatttgtaccagccttttaaaaagttttagtttttcctaagtgattttgctctcttccaatctaaacctgttgcttgtttggttcagagaactacaaactgtcaaagaaagggtggggatgataagaaatgctaatataaaaatgctaagtgaaaaaaagacttggccaggagaaataatttaaaatgcacatttgctttggatgcactgttgttctgttaaggctgtatatatttgtttatttaaggtgactgaaagtgcaaagaggaaatggacagcatgcaattcatcctaatgtacaaaacgttatatgcactcaaatgttataatttctaatatttttaaagtttatattcgagttgtacaaagttaagcattaatcagatatttcattttttcataatgttaccattttcttaaatattattacaaaattttaagtctgtctaatggagagttttttttaaactgtctacctcatataatacaagtatttacaacgctaaagttaccagaggtcaatgaataatcaaaacattttttacagtacacctttcctggatgatatgcaatcgaatgctatattattaaacgcatttttctccttattaaaaaaaaaaaaaaaaa

SEQ ID NO: 17 - protein, 450 aa, Homo sapiens
MDLSAAAALCLWLLSACRPRDGLEAAAVLRAAGAGPVRSPGGGGGGGGGGRTLAQAAGAAAVPAAAVPRARAARRAAGSGFRNGSVVPHHFMMSLYRSLAGRAPAGAAAVSASGHGRADTITGFTDQATQDESAAETGQSFLFDVSSLNDADEVVGAELRVLRRGSPESGPGSWTSPPLLLLSTCPGAARAPRLLYSRAAEPLVGQRWEAFDVADAMRRHRREPRPPRAFCLLLRAVAGPVPSPLALRRLGFGWPGGGGSAAEERAVLVVSSRTQRKESLFREIRAQARALGAALASEPLPDPGTGTASPRAVIGGRRRRRTALAGTRTSQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 18 - DNA, 1994 bp, Homo sapiens
ctttgaggccgccgggagcatcctgtggcctctctctgcgcggccacccggccgcggcgcgaagcggtctggagggcgagcccttccgcggccccaactctgccgccccgttcccggcattgggaaccagggcagggagggggcgggtgtttctctgcgggggagtggggaggaagctgggcgggtgcgcgcggtgccccgagcctggaaccacggagggcgcgttggtcttgggcggatggagggggtgtcgcactgccgcggggaggcgtgtcgggaggctggggccagtggcagtcgcttggcgagggtgggggcgtagcgctgcggtgggaggaggcggctccggccctggtctccactctaggccggggtggggggcgcatagcggccgccggagctttcagcagggggcgctgctccgggcgttgggcgggggtggggtgggccaggagggggggccgcgggctggccgcgcacacttcccccattattaaacactatgttcaaaaggcgccgggggacttcccggagccacggagcccgcgccgcccgcccgcccggcccacggagcccatggacctgagcgccgccgccgcgctgtgcctttggctgctgagcgcctgccgcccccgcgacgggctggaagcggccgccgtgctgcgagcggcgggggctgggccggtccggagcccagggggcggcggcggcggcggcggcggcgggcggactcttgcccaggctgcgggcgccgcggctgtcccggccgccgcggttccccgggcccgcgccgcgcgccgcgccgcgggctccggcttcaggaacggctcggtggtgccgcaccacttcatgatgtcgctttaccggagcctggccgggagggctccggccggggcagccgctgtctccgcctcgggccatggtcgcgcggacacgatcaccggcttcacagaccaggcgacccaagacgaatcggcagccgaaacaggccagagcttcctgttcgacgtgtccagccttaacgacgcagacgaggtggtgggtgccgagctgcgcgtgctgcgccggggatctccagagtcgggcccaggcagctggacttctccgccgttgctgctgctgtccacgtgcccgggcgccgcccgagcgccacgcctgctgtactcgcgggcagctgagcccctagtcggtcagcgctgggaggcgttcgacgtggcggacgccatgaggcgccaccgtcgtgaaccgcgccccccccgcgcgttctgcctcttgctgcgcgcagtggcaggcccggtgccgagcccgttggcactgcggcggctgggcttcggctggccgggcggagggggctctgcggcagaggagcgcgcggtgctagtcgtctcctcccgcacgcagaggaaagagagcttattccgggagatccgcgcccaggcccgcgcgctcggggccgctctggcctcagagccgctgcccgacccaggaaccggcaccgcgtcgccaagggcagtcattggcggccgcagacggaggaggacggcgttggccgggacgcggacatcgcagggcagcggcgggggcgcgggccggggccacgggcgcaggggccggagccgctgcagccgcaagccgttgcacgtggacttcaaggagctcggctgggacgactggatcatcgcgccgctggactacgaggcgtaccactgcgagggcctttgcgacttccctttgcgttcgcacctcgagcccaccaaccatgccatcattcagacgctgctcaactccatggcaccagacgcggcgccggcctcctgctgtgtgccagcgcgcctcagccccatcagcatcctctacatcgacgccgccaacaacgttgtctacaagcaatacgaggacatggtggtggaggcctgcggctgcaggtagcgcgagggccggggagggggcagccacgcggccgaggatccccagctgatgagcagcagcgggcc